(19) United States
(12) Patent Application Publication
(10) Pub. No.: US 2025/0319017 A1
(43) Pub. Date: Oct. 16, 2025

(54) OPEN-CELL FOAM BASED WOUND TREATMENT

(71) Applicant: Scott C. Smith, Osterville, MA (US)

(72) Inventor: Scott C. Smith, Osterville, MA (US)

(21) Appl. No.: 18/869,055

(22) PCT Filed: May 25, 2023
(86) PCT No.: PCT/US2023/023617

§ 371 Date: Nov. 25, 2024

(51) Int. Cl. A61K 9/00; A61K 9/70; A61K 33/00; A61K 33/02; A61K 33/20; A61K 33/40; A61P 31/02

(52) U.S. Cl. CPC A61K 9/0014; A61K 9/70; A61K 33/00; A61K 33/02; A61K 33/20; A61K 33/40; A61P 31/02

(57) ABSTRACT

Disclosed herein include methods of killing or preventing growth of one or more biological contaminants and/or pathogens on or in a subject.

### PRIORITY CLAIM

This application claims the benefit of U.S. Provisional Application No. 63/345,836, filed May 25, 2022, which is incorporated by reference herein in its entirety.

### TECHNICAL FIELD

This invention relates to disinfectants, articles comprising one or more disinfectants, and their preparation and use.

### BACKGROUND

It is desirable to kill pathogens to prevent and/or treat wound infections. Novel methods provided herein show potentials in preventing and/or treating conditions associated with pathogens, e.g., without limitation, surgical site infections (SSI) whose most common cause is Staphylococcus aureus.

### SUMMARY

One aspect of the present disclosure relates to methods of killing one or more biological contaminants and/or pathogens on or in a subject comprising contacting the subject with an article, and contacting the one or more biological contaminants and/or pathogens with one or more biocides and/or disinfectants. In certain embodiments, one or more of the one or more biological contaminants and/or pathogens are on a surface (e.g., without limitation, the skin) or in a wound of the subject. In certain embodiments, the one or more biocides and/or disinfectants are applied to the article before, during, and/or after the article contacting the subject. In certain embodiments, the one or more biocides and/or disinfectants are applied to the subject before, during, and/or after the article contacting the subject.

Another aspect of the disclosure provided herein relates to methods of remediating an exposure of one or more biological contaminants and/or pathogens to a subject comprising contacting the subject at or near a location of the exposure with an article before, during, and/or after the exposure, and contacting the one or more biological contaminants and/or pathogens with one or more biocides and/or disinfectants. In certain embodiments, the one or more biocides and/or disinfectants are applied to the article before, during, and/or after the article contacting the subject. In certain embodiments, the one or more biocides and/or disinfectants are applied to the subject before, during, and/or after the article contacting the subject.

Another aspect of the disclosure provided herein relates to methods of preventing or treating a wound infection of a subject comprising contacting the wound with an article, and contacting the article with one or more biocides and/or disinfectants before, during, and/or after the article contacting the wound. In certain embodiments, the one or more biocides and/or disinfectants are applied to the subject before, during, and/or after the article contacting the subject.

Another aspect of the disclosure provided herein relates to methods of preventing a wound infection of a subject comprising contacting the subject at or near a location of the wound with an article before the wound is created, and contacting the article with one or more biocides and/or disinfectants before, during, and/or after the article contacting the subject. In certain embodiments, the one or more biocides and/or disinfectants are applied to the subject before, during, and/or after the article contacting the subject.

In certain embodiments of the methods disclosed herein, the article comprises one or more foam structures. In certain embodiments of the methods disclosed herein, each of the one or more foam structures comprises one or more open-cell foam structures. In certain embodiments of the methods disclosed herein, at least one of the one or more foam and/or open-cell foam structures has an open cell structure with an average cell size of about 0.3 mm to about 1.7 mm, about 0.4 mm to about 1.5 mm, about 0.5 mm to about 1.3 mm, about 0.6 mm to about 1.2 mm, about 0.7 mm to about 1.0 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, or about 1.7 mm. In certain embodiments, the foam and/or open-cell foam structures have a density range of about 1.5 pcf to about 3.0 pcf, about 1.5 pcf to about 2.6 pcf, about 1.6 pcf to about 2.2 pcf, about 1.7 pcf to about 2.0 pcf, about 1.5 pcf, about 1.6 pcf, about 1.7 pcf, about 1.8 pcf, about 1.9 pcf, about 2.0 pcf, about 2.1 pcf, about 2.2 pcf, about 2.3 pcf, about 2.4 pcf, about 2.5 pcf, about 2.6 pcf, about 2.7 pcf, about 2.8 pcf, about 2.9 pcf, or about 3.0 pcf.

In certain embodiments of the methods disclosed herein, the article comprises an open-cell foam which is infused with one or more of the one or more biocides and/or disinfectants prior to being applied to the subject.

In certain embodiments of the methods disclosed herein, the wound is a wound on the skin of the subject. In certain embodiments of the methods disclosed herein, the wound is a surgical site.

In certain embodiments of the methods disclosed herein, the wound infection is associated with one or more biological contaminants and/or pathogens. In certain embodiments of the methods disclosed herein, the wound infection is SSI.

In certain embodiments of the methods disclosed herein, the article may comprise an open-cell foam structure, e.g., AquaFlex Open-Cell Foam. In certain embodiments of the methods disclosed herein, the biocide or disinfectant comprises a hydronium composition. In certain embodiments of the methods disclosed herein, the hydronium composition has a pH of about 1.2 to about 6.0. In certain embodiments, the hydronium composition comprises water and hydronium, including one or more hydrates thereof. Examples of hydrates of hydronium include, without limitation, hydrates with one water molecule (H₅O₂⁺), two water molecules (H₇O₃⁺), and three water molecules (H₉O₄⁺). In certain embodiments, a hydronium composition is prepared by adding a strong acid into water until a desired pH is reached. Examples of the strong acid include, without limitation, sulfuric acid and hydrochloric acid.

In certain embodiments of the methods disclosed herein, at least one of the biological contaminants and/or pathogens is a bacteria (e.g., ESKAPE pathogens, Enterococcus faecium, Staphylococcus aureus, Klebsiella pneumoniae, Acinetobacter baumannii, Pseudomonas aeruginoa, and Enterobacter spp.). In certain embodiments of the methods disclosed herein, at least one of the biological contaminants and/or pathogens is a virus (e.g., SARS-CoV-2).

Other aspects of the present disclosure relate to disinfectants (e.g., without limitation, hydronium compositions), and articles carrying one or more biocides and/or disinfectants. In certain embodiments, the articles comprise or are made of one or more foam structures which may comprise one or more open-cell foam structures. In certain embodiments, the article is a sponge, bandage, or applicator, e.g., for wound care.

### BRIEF DESCRIPTION OF THE DRAWINGS

FIG. 1: The inhibitory sigmoid Emax curve of Staphylococcus aureus (log₁₀ CFU/mL) versus hydronium concentration (provided in fold dilution) showing mean bacterial burden with standard deviation.

### DETAILED DESCRIPTION

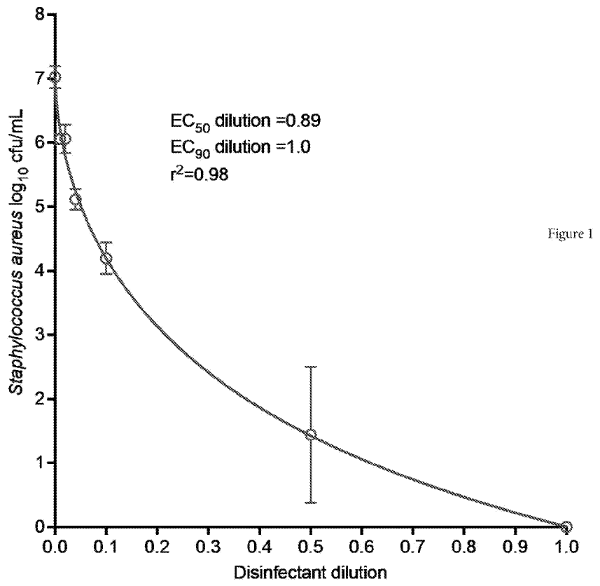

The potential corrosive property of hydronium limits its application as a disinfectant on living subjects. Provided in the Example section are examples of hydronium compositions prepared from a hydronium solution in water (pH 1.6) that effectively reduced bacterial burden of Staphylococcus aureus, which is the most common cause of surgical site infection (SSI), which accounts for 20% of all SSI infections (Example 1, FIG. 1). The hydronium compositions were not corrosive and may be applied to subjects like human and animals.

One aspect of the present disclosure relates to methods of killing one or more biological contaminants and/or pathogens on or in a subject comprising contacting the subject with an article, and contacting the one or more biological contaminants and/or pathogens with one or more biocides and/or disinfectants. In certain embodiments, one or more of the one or more biological contaminants and/or pathogens are on a surface (e.g., without limitation, the skin) or in a wound of the subject. In certain embodiments, the one or more biocides and/or disinfectants are applied to the article before, during, and/or after the article contacting the subject.

Another aspect of the disclosure provided herein relates to methods of remediating an exposure of one or more biological contaminants and/or pathogens to a subject comprising contacting the subject at or near a location of the exposure with an article before, during, and/or after the exposure, and contacting the one or more biological contaminants and/or pathogens with one or more biocides and/or disinfectants. In certain embodiments, the one or more biocides and/or disinfectants are applied to the article before, during, and/or after the article contacting the subject.

Another aspect of the disclosure provided herein relates to methods of preventing or treating a wound infection of a subject comprising contacting the wound with an article, and contacting the article with one or more biocides and/or disinfectants before, during, and/or after the article contacting the wound.

Another aspect of the disclosure provided herein relates to methods of preventing a wound infection of a subject comprising contacting the subject at or near a location of the wound with an article before the wound is created, and contacting the article with one or more biocides and/or disinfectants before, during, and/or after the article contacting the subject.

In certain embodiments of the methods disclosed herein, the wound is a wound on the skin of the subject. In certain embodiments of the methods disclosed herein, the wound is a surgical site.

In certain embodiments of the methods disclosed herein, the wound infection is associated with one or more biological contaminants and/or pathogens. In certain embodiments of the methods disclosed herein, the wound infection is SSI.

In certain embodiments of the methods disclosed herein, at least one of the biological contaminants and/or pathogens is a bacteria (e.g., Staphylococcus aureus). In certain embodiments of the methods disclosed herein, at least one of the biological contaminants and/or pathogens is a virus (e.g., SARS-CoV-2).

Examples of pathogens include, without limitation, microbiological organisms (e.g., without limitation, bacteria, viruses, fungus, parasitic flatworms, and protozoa), biological organisms, and biological contaminants. Examples of bacteria include, without limitation, ESKAPE pathogens, Enterococcus faecium, Staphylococcus aureus, Klebsiella pneumoniae, Acinetobacter baumannii, Pseudomonas aeruginoa, and Enterobacter spp., Vibrio cholerae, Salmonella, Clostridium, Campylobacter, Legionella, and E. coli. Examples of viruses include, without limitation, SARS-CoV-2, H1N1, norovirus, rotavirus, and Enteroviruses. Examples of parasitic flatworms include, without limitation, Schistosoma. Examples of protozoa include, without limitation, protozoan cysts and protozoan parasites.

Examples of the surfaces of a subject include, without limitation, the skin of the subject.

Examples of the subject include, without limitation, humans and other non-human animals. Examples of non-human animals include, without limitation, pet (e.g., cats, dogs), cows, bulls, horses, and sheep.

The articles can be fabricated into a number of different forms to suit the particular use scenario or application. Examples of the different forms include, without limitation, bandage, and applicators, e.g., for wound care.

In certain embodiments of the methods disclosed herein, the article comprises one or more foam structures. In certain embodiments of the methods disclosed herein, each of the one or more foam structures comprises one or more open-cell foam structures.

In certain embodiments of the methods disclosed herein, the articles comprising or made of the foam and/or open-cell foam structures can be wiped or pushed over a surface to remediate the surface from exposure to the one or more biological contaminants and/or pathogens, a process that is sometimes called “swabbing.” Swabbing can pick up pathogens and biological contaminants that are on the surface that is swabbed. Swabbing can also pick up other contaminants.

In certain embodiments of the methods disclosed herein, the article is applied with the one or more biocides and/or disinfectants before, during, or after the contact of the article with the one or more biological contaminants and/or pathogens.

In certain embodiments of the methods disclosed herein, the foam and/or the open-cell foam structure can be further applied with one or more other chemicals or substances that can kill or neutralize biological contaminants and/or pathogens, which include bacteria, viruses, or other microbiological or biological organisms and other biological contaminants.

Examples of biocides and disinfectants include, without limitation, citric acid, hydrogen peroxide, peracetic acid, hypoclorous acid, hydronium, and quat.

In certain embodiment, the biocide comprises hydronium compositions comprising hydronium, including hydrates thereof (e.g., hydrates with one water molecule (H₅O₂⁺), two water molecules (H₇O₃⁺), and three water molecules (H₉O₄⁺). In certain embodiments of the methods disclosed herein, the hydronium composition has a pH of about 1.2 to about 6.0, about 1.2 to about 5.0, about 1.2 to about 4.0, about 1.2 to about 3.0, about 1.2 to about 2.0, about 1.2 to about 1.8, about 1.2, about 1.6, about 1.8, about 2.0, about 3.0, about 4.0, about 5.0, or about 6.0. In certain embodiments, the hydronium composition comprises water and hydronium, including one or more hydrates thereof. In certain embodiments, the hydronium composition is not corrosive to the subject.

In certain embodiments, the biocides and/or disinfectants may comprise chemicals that carry positive charges, e.g., quat and hydronium. In certain embodiments, the biocides and/or disinfectants may comprise chemicals that carry negative charges. In certain embodiments, the biocides and/or disinfectants may comprise chemicals that have net neutral charges. In certain embodiments, the biocides and/or disinfectants may have neutral charges. In certain embodiments, the biocides and/or disinfectants may comprise chemicals that carry no charges.

In certain embodiments of the methods disclosed herein, the article comprises an open-cell foam which is infused with one or more of the one or more biocides and/or disinfectants prior to being applied to the subject.

In certain embodiments of the methods disclosed herein, contacting the article with the subject comprises contacting the subject with at least one of the one or more foam and/or open-cell foam structures in the presence of at least one of the one or more biocides and/or disinfects. In certain embodiments, the contact of the foam and/or open-cell foam structures with the subject improves contact of the one or more biocides and/or disinfects with the one or more biological contaminants and/or pathogens.

Other aspects of the present disclosure relate to disinfectants (e.g., without limitation, hydronium compositions), and articles carrying one or more disinfectants. In certain embodiments, the articles comprise or are made of one or more foam structures which may comprise one or more open-cell foam structures.

Physical Characteristics of the Foam and/Open-Cell Foam Structures

In certain embodiments, the foam and/or open-cell foam structures may be hydrophobic. In certain embodiments, the foam and/or open-cell foam structures may be antimicrobial. In certain embodiments, the foam and/or open-cell foam structures may have substantially no ionic charge. In certain embodiments, the foam and/or open-cell foam structures may have a high surface area. In certain embodiments, the foam and/or open-cell foam structures may have a density lower than that of water. In certain embodiments of the methods disclosed herein, at least one of the one or more foam and/or open-cell foam structures has an open cell structure with an average cell size of about 0.3 mm to about 1.7 mm. In certain embodiments of the methods disclosed herein, at least one of the one or more foam and/or open-cell foam structures has a density range of about 1.5 pcf-to about 3.0 pcf,

The foam and/or the open-cell foam structure may be hydrophobic and therefore repels water. The foam and/or the open-cell foam structure that is hydrophobic may be antimicrobial without additional additives. In certain embodiments, the foam and/or the open-cell foam structure are made from one or more polymers that are oleophilic. The oleophilic nature of the constituent polymer(s) prevents the absorption of water and promotes absorption and adsorption of oils and other organic substances such as pathogens and viruses. While we do not wish to be bound by theory, the foam and/or the open-cell foam structures may increase the efficiency of release of biocides and/or disinfectants in aqueous compositions (e.g., hydronium aqueous solutions) loaded in the foam and/or the open-cell foam structures.

In certain embodiments, the foam and/or the open-cell foam structure may have substantially no ionic charge. Accordingly, the foam and/or the open-cell foam having substantially no ionic charge is not likely to impact the efficacy of biocides and/or disinfectants comprising cations (e.g., hydronium, and quat) or anions. While we do not wish to be bound by theory, it is known/proven that during the recent pandemic of 2020, paper towels, cotton rags, and cellulose sponges having negative ionic charges may have deactivated disinfectants (e.g. quats) have a positive ionic charge thereby cancelling each other out and rendering the disinfectant inactive leaving live pathogens (e.g., virus such as SARS-CoV-2) on surfaces. Deactivation of disinfectant in this manner is sometimes referred to as “quat binding.” The phenomenon of quat binding occurs when the active ingredient (quaternary ammonium chloride) becomes attracted to and absorbed into fabrics. The science behind how this happens is simple: Quats are positively charged ions and cotton and other natural textiles are negatively charged; positive attracts negative. In United States Patent Publication US 2007/0142261 A1 entitled, “WIPER FOR USE WITH DISINFECTANTS” (incorporated by reference herein), the problems with quat binding and deactivation of disinfectant are well established. In certain embodiments, the foam and/or the open-cell foam structure having substantially no ionic charge comprises one or more non-polar polymers and/or substantially non-polar polymers. In certain embodiments, biocidal effects of the one or more biocides and disinfectant after their contact with the article are at least about 95%, at least about 90%, at least about 85%, or at least about 80% of their biocidal effects before contacting the article.

In certain embodiments, the foam and/or the open-cell foam structures do not comprise co-polymers comprising vinyl acetate or methyl acrylate or other polar co-polymers. In certain embodiments, the foam and/or the open-cell foam structures may carry ionic charge(s).

The open-cell foam structure provides a high surface area due to the interconnected structure of the individual cells. In certain embodiments, for a 1″ thick×12″×12″ open-cell foam, ranges of surface area may be from about 200 square feet to about 500 square feet, about 300 square feet to about square 500 feet, about 400 square feet to about square 500 feet, or about 450 square feet to about 500 square feet. The open-cell structure of the open-cell foam foams behaves similar to the alveoli of the human lungs in that it increases surface area which increases the ability of the open-cell foam structure to carry biocides and/or disinfectants, as well as picking up biological and related contamination at the molecular level for more effective removal. The high surface area for absorption and release of active disinfectant substantially mitigates the risk of gaps in surface coverage or not making contact with live pathogens on surfaces. In certain embodiments, the high surface area combined with a neutral or substantially neutral ionic charge, substantially limits deactivation of disinfectants/biocides along with maximum delivery of disinfectants/biocides per square inch of surface area.

In certain embodiments, the foam and/or open-cell foam structure has a density in the range of from about 1.0 pcf (pounds per cubic foot) to about 50.0 pcf. In certain embodiments, the foam and/or open-cell foam structure has a density of less than the specific gravity of water (62.3 pcf at 70° F.). In certain embodiments, the foam and/or open-cell foam structures have a density range of about 1.5 pcf to about 3.0 pcf, about 1.5 pcf to about 2.6 pcf, about 1.6 pcf to about 2.2 pcf, about 1.7 pcf to about 2.0 pcf, about 1.5 pcf, about 1.6 pcf, about 1.7 pcf, about 1.8 pcf, about 1.9 pcf, about 2.0 pcf, about 2.1 pcf, about 2.2 pcf, about 2.3 pcf, about 2.4 pcf, about 2.5 pcf, about 2.6 pcf, about 2.7 pcf, about 2.8 pcf, about 2.9 pcf, or about 3.0 pcf.

In certain embodiments of the methods disclosed herein, at least one of the one or more foam and/or open-cell foam structures has an open cell structure with an average cell size of about 0.3 mm to about 1.7 mm, about 0.4 mm to about 1.5 mm, about 0.5 mm to about 1.3 mm, about 0.6 mm to about 1.2 mm, about 0.7 mm to about 1.0 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, or about 1.7 mm.

Chemical Characteristics and Preparation of the Foam and/or Open-Cell Foam Structures

The foam and/or open-cell foam structures can be extruded or produced in a bun/batch process. The foam and/or open-cell foam structures can be crosslinked or non-crosslinked. Also, the foam and/or open-cell foam structures can utilize either physical blowing agents or chemical blowing agents. Furthermore, a bio-degradable initiator may be added to the foam and/or open-cell foam structures so that after use the foam and/or open-cell foam structures will degrade over time in a landfill environment when disposed.

In certain embodiments, the foam and/or open-cell foam structures can comprise and/or be made from one or more polymers. In certain embodiments, the foam and/or the open-cell foam structures comprise or are made from one or more medical grade polymers and plasticizer free. In certain embodiments, the base polymer used in the manufacture of the foam and/or the open-cell foam structure has a specific gravity of about 0.875, or about 0.85 to about 0.90 before foaming.

In one non-limiting example, the foam and/or open-cell foam structures are produced from an ethylene and alkyl acrylate copolymer or an ethylene vinyl acetate copolymer.

In another non-limiting example, the foam and/or open-cell foam structures are produced without plasticizer from a single site initiated/metallocene catalyzed polyolefin polymer or a single site initiated/metallocene catalyzed polyolefin polymer blended with other polymers, e.g., without limitation, a low density polyethylene polymer or copolymer. In certain embodiments, a blend used for producing the foam and/or open-cell foam structures is LDPE with a single site initiated/metallocene catalyzed polyolefin polymer. The foam and/or open-cell foam structures can comprise an elastomeric polyolefin. Examples of elastomeric polyolefins include, without limitation, ethylene methyl acrylate (EMA), and a single site initiated polyolefin elastomer (e.g. Dow or DuPont Dow Engage 8452). The foam and/or open-cell foam structures may also be composed of a polyolefin elastomer which includes, without limitation, relatively amorphous elastomers and/or blends of other polymers.

The foam and/or open-cell foam structures can be fabricated from a very specific formulation for an open-cell foam. Specifically, this open-cell foam may be produced from 80-100% ethylene acrylate copolymer. Blends of LDPE (low density polyethylene) with ethylene acrylate copolymer can be used also. See, e.g., U.S. Pat. No. 8,853,289 and US Patent Publication No. 2013/0240451 A, for additional embodiments of open cell foam structures, the disclosure of which is incorporated herein by reference.

A substantially non-polar open-cell foam structure may be prepared by 80-100% EMA (ethylene methyl acrylate).

Additional open-cell foam structures may be produced from one or more polymers including but not limited to polyurethane, ethylene-ethyl acrylate (EEA), ethylene-butyl acrylate (EBA), EVA (ethyl vinyl acetate), EPDM (ethylene propylene diene monomer), elastomers, polyolefin elastomers, low density polyethylene (LDPE), linear low density polyethylene (LLDPE), high density polyethylene (HDPE), polypropylene (PP), neoprene, styrene butadiene rubber, ionic co-polymers, other synthetic rubbers, natural rubber, chlorinated polyethylene (CPE), olefin block copolymers, ethylene maleic anhydride copolymer, very low density polyethylene (VLDPE), singe site initiated polyolefins, metallocene catalyzed polyolefins, silane-modified polymers (including but not limited to silane grafted, silane functionalized, and silane cross-linked polymers), maleic anhydride grafted polymers, styrene-butadiene-styrene copolymers, polyisoprene, and equivalents to any and all of these polymers. Silane modification of polymers can occur during the manufacturing process of the foam and/or open-cell foam structures, or as a separate step after the foaming process, e.g., the silane can be applied in liquid form post-foaming. Further, specific silane-modified polymers may be tailored to target specific contaminants that may be present in water with bacteria, such as VOCs and SVOCs related to oils and/or industrial chemicals, metals (e.g. copper, iron, etc.) or metalloids (e.g. phosphorus) and other petroleum products, and surfactants, including but not limited to methylene blue active substances (MBAS).

In certain embodiments, the method of killing one or more biological contaminants and/or pathogens comprises providing an article that comprises an open-cell foam structure, impregnating the open-cell foam with a substance that comprises one or more biocides and/or other chemicals that can kill biological contaminants and/or pathogens, and exposing the article to the one or more biological contaminants and/or pathogens such that the impregnated open-cell foam contacts and kills the one or more biological contaminants and/or pathogens.

In certain examples, the one or more biological contaminants and/or pathogens are on a surface such as the skin surface of a subject. In certain examples, exposing the article to the one or more biological contaminants and/or pathogens comprises moving the article over the surface such that the impregnated open-cell foam contacts the one or more biological contaminants and/or pathogens, removes the one or more biological contaminants and/or pathogens from the surface, and kills the one or more biological contaminants and/or pathogens.

In certain embodiments, the method of remediating an exposure of one or more biological contaminants and/or pathogens that are on a surface includes providing an article that comprises an open-cell polymer foam structure, applying one or more biocides and/or disinfectants that can kill the one or more biological contaminants and/or pathogens to one or more of the sides of the open-cell polymer foam structure, and moving the article over the surface such that the open-cell foam structure contacts the one or more biological contaminants and/or pathogens, removes the one or more biological contaminants and/or pathogens from the surface, and the one or more biological contaminants and/or pathogens are killed. In certain examples, the one or more biocides and/or disinfectants comprise at least a hydronium-based biocide/disinfectant and another chemical-based disinfectant (e.g., citric acid, hydrogen peroxide, peracetic acid, hypochlorous acid, and quats).

In certain embodiments, the method of remediating an exposure of one or more biological contaminants and/or pathogens comprises providing an article that comprises an open-cell foam structure, contacting the article to a skin surface (e.g., by placing the article over a wound) such that the open-cell foam contacts the one or more biological contaminants and/or pathogens, and either before or after the contacting step, applying to the open-cell foam structure one or more biocides and/or disinfectants that can kill the one or more biological contaminants and/or pathogens, to kill the one or more biological contaminants and/or pathogens that come into contact with the open-cell foam structure. In certain examples, the one or more biocides and/or disinfectants comprise at least one of a chemical-based disinfectant and a hydronium-based disinfectant. In certain examples, one or more of the one or more biocides and/or disinfectants are applied after the contacting step. In some examples, the method further comprises wringing out the open-cell foam structure after the application of the one or more biocides and/or disinfectant, to remove at least some of the one or more biocides and/or disinfectants from the open-cell foam structure. In certain examples, the method further comprises reusing the wrung-out open-cell foam structure by contacting the open-cell foam structure a second time to the surface such that the open-cell foam structure is put in contact with the one or more biological contaminants and/or pathogens. In certain examples, the application of the one or more biocides and/or disinfectants comprises impregnating the open-cell foam structure with the one or more biocides and/or disinfectants.

The above disclosure and the following example are illustrative of the teachings of this application and are not meant to limit the scope and application of the invention. The examples and embodiments are for illustrative and will suggest modifications to persons skilled in the art which are included within the spirit and purview of this application and scope of the appended claims.

### EXAMPLES

### Example 1. Dose-Response Curves of Hydronium Versus Staphylococcus aureus

The most common cause of surgical site infection [SSI] is Staphylococcus aureus, which accounts for 20% of all SSI infections. Standard Pharmacokinetic/Pharmacodynamic definitions to measure efficacy require testing of several doses and establishing a dose response curve using the inhibitory sigmoid Emax model.

Staphylococcus aureus was grown to logarithmic growth phase to a bacterial density of 10⁷ colony forming units per mL [CFU/mL], or 7.0 log 10 CFU/mL at 37° C. under shaking conditions in cation-adjusted Mueller-Hinton broth [CAMHB]. A hydronium solution in water (pH 1.6) was diluted in saline and then 0.2 mL added to 1.8 mL of cultures to make a final dilution of 1:1, 1:10, 1:25, 1:50, 1:100 which means 0.5, 0.1, 0.04, 0.02, 0.01-fold, as well as not diluted (1.0). The hydronium and Staphylococcus aureus were then co-incubated for 10 minutes at 37° C., after which the cultures were centrifuged and supernatant decanted, and resuspended in saline, twice, to remove hydronium carryover. There were two replicates for each condition. For analysis we used the Pharmacokinetics/Pharmacodynamics inhibitory sigmoid Emax model of log₁₀ CFU/mL versus hydronium dilution.

Results:

Under non-diluted concentrations hydronium wiped out all 10,000,000 CFU/mL of Staphylococcus aureus, as shown in FIG. 1. The inhibitory sigmoid Emax parameters were an Econ of 7.02 log₁₀ CFU/mL [which is bacterial burden in cultures not exposed to hydronium], Emax or efficacy >7.02 log₁₀ CFU/mL [which is the maximal effect or microbial kill of hydronium against Staphylococcus aureus], a Hill slope of 0.61, and an EC₅₀ or potency of 0.89-fold [which is the concentration or dose mediating 50% of Eₘₐₓ]; the r2 was 0.98 indicating excellent fit of the model to the data. This means hydronium was effective in killing the common SSI pathogen, Staphylococcus aureus.

FIG. 1 depicts the inhibitory sigmoid Eₘₐₓ curve of Staphylococcus aureus log₁₀ CFU/mL versus hydronium concentration [presented using fold dilution]. Symbols are mean bacterial burden and error bars are standard deviation.

What is claimed is:

1. A method of killing one or more of biological contaminants and pathogens on or in a skin or wound location of a subject, comprising:
contacting the location of the subject with an article comprising an open-cell foam structure; and
contacting the one or more of the biological contaminants and pathogens with at least one of a biocide and a disinfectant;
wherein the at least one of a biocide and a disinfectant is applied to at least one of the article and the skin or wound location of the subject, at least one of before, during, and after the article contacts the skin or wound location of the subject.

2. The method of claim 1, wherein the open-cell foam structure has an open cell structure with an average cell size of about 0.3 mm to about 1.7 mm.

3. The method of claim 1, wherein the open-cell foam structure has a density range of about 1.5 pcf to about 3.0 pcf.

4. The method of claim 1, wherein the wound is a wound on the skin of the subject.

5. The method of claim 1, wherein the at least one of a biocide and a disinfectant comprises a hydronium composition.

6. The method of claim 5, wherein the hydronium composition has a pH of about 1.2 to about 6.0.

7. The method of claim 5, wherein the hydronium is prepared by adding a strong acid to water.

8. The method of claim 7, wherein the strong acid is at least one of sulfuric acid and hydrochloric acid.

9. The method of claim 1, wherein at least one of the biological contaminants and pathogens is a bacteria or virus.

10. The method of claim 9, wherein the bacteria are selected from the group consisting of Enterococcus faecium, Staphylococcus aureus, Klebsiella pneumoniae, Acinetobacter baumannii, Pseudomonas aeruginoa, Enterobacter spp., and any combination thereof.

11. The method of claim 9, wherein the virus is SARS-CoV-2.

12. The method of claim 1, wherein the article is a sponge, bandage, or applicator.

13. The method of claim 1, wherein the open-cell foam structure has one or more properties selected from the group consisting of non-polar, antimicrobial, having no charge, and having a net neutral charge.

14. The method of claim 1, wherein biocidal effects of the at least one of a biocide and a disinfectant after the contact with the article are at least about 95%, at least about 90%, at least about 85%, or at least about 80% of their biocidal effects before contacting the article.

15. The method of claim 1, wherein the at least one of a biocide and a disinfectant comprises at least one of citric acid, hydrogen peroxide, peracetic acid, hypochlorous acid, hydronium, and quaternary ammonium chloride (quats).
